(19) United States
(12) Patent Application Publication
(10) Pub. No.: US 2008/0279792 A1
(43) Pub. Date: Nov. 13, 2008

(54) Cosmetic And Dermatological Self-Tanning Formulations Comprising Dihydroxyacetone And Glycerin

(75) Inventors: Anja Eitrich, Hamburg (DE); Rixa Dippe, Hamburg (DE); Tobias Mann, Hamburg (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 10/589,084

(22) PCT Filed: Jan. 13, 2005
(86) PCT No.: PCT/EP05/50134

§ 371 Date: Oct. 4, 2006

(30) Foreign Application Priority Data
Feb. 11, 2004 (DE) 10 2004 006.830.5

(51) Int. Cl. A61K 8/35

(52) U.S. Cl. 424/59

(57) ABSTRACT

Cosmetic or dermatological self-tanning preparations which contain dihydroxyacetone and are characterized in that they also contain more than 5 percent by weight (relative to the total weight of the preparation) of glycerin, and the use thereof.

The present invention relates to cosmetic and dermatological self-tanning preparations with a content of dihydroxyacetone and glycerin.

Although natural tanning through the effect of solar rays is the most dangerous method, it continues to be the most common and most favored method of achieving desired pigmentation of the skin. The sun can be enjoyed and exploited virtually everywhere: during a beach holiday at the coast, during skiing, during hill walking, during freetime sport and also even during brief sunbathing during the lunch break. Sunbathing—enjoyed in moderation—has an effect which is pleasantly relaxing, calming and recuperative. It improves the disposition and mood. Not to be forgotten are the positive effects on the entire organism via stimulation of various metabolic processes.

However, solar rays also emanate a series of disadvantageous effects: the harmful effect of the ultraviolet part of solar radiation on the skin is generally known. Depending on their particular wavelength, the rays have various effects on the skin organ. Accordingly, many consumers have become more careful where the sun is concerned. Their attitude to sunbathing has changed: tanned at any cost is out—healthy tan is in.

Artificial tanning is understood as meaning all measures which impart a tanned appearance to the skin without the effect of solar rays. Several methods are in principle conceivable for this purpose:
The simplest method is the application of make-up. These are dye preparations which merely adhere physically to the surface of the skin, in contrast to the self-tanning preparations, which form a true chemical bond with constituents of the horny layer.
A further method of artificial tanning is the taking of carotenoids in the form of capsules or dragees, which leads to a yellowish-brown skin coloration.
A third method of artificial tanning is irradiation of the skin using technically manufactured light sources which are able to emit ultraviolet rays of varying type and intensity.

Whereas tanning the skin with ultraviolet rays, as occur in the natural solar spectrum or are generated by artificial light sources, can lead to not inconsiderable skin damage upon excessively long exposure and exceeding a certain dose, the use of make-up, self-tanning creams and tanning pills containing beta-carotene can be regarded as harmless and without risk. Someone who thus wishes to avoid light-induced skin damage, such as sunburn, chronic photo damage or possible risk of carcinoma, but does not want to go without the fashionably desirable tan has to rely on those methods of tanning which work without the use of artificially generated or natural ultraviolet rays. The same is true for people who must not be exposed to ultraviolet light on account of pathological skin reactions.

The fact that some substances take part in a chemical color reaction with the keratin of (animal) skin, hair and wool was already known from textile chemistry and vat dyeing. Finally, for cosmetic application, a class of active ingredients which meet the requirements of modern cosmetic compositions has gained acceptance. These active ingredients are nonirritative, easy to handle, and the resulting shade is lighffast and can not be washed off.

Use is made of substances with an adjacent keto-alcohol or aldehyde-alcohol group, i.e. ketols or aldols which primarily belong to the class of sugars. The basic material which is most important and continues to be used most frequently is dihydroxyacetone (DHA), a trivalent sugar which occurs in the human body. DHA reacts with the proteins and amino acids of the horny layer, where the amino group of the amino acids reacts with the keto group of the DHA in the sense of a Maillard reaction (Schiff's base). The shade arises as a result of a polymerization whose individual reaction steps have still not been completely clarified.

This type of tanning is a purely chemical reaction of the horny layer and has nothing to do with the formation of new melanin. The colored products arising with DHA on the keratin are referred to as melanoids. Experiments have shown that the tanning properties of DHA can be influenced considerably by the formulation: thus, for example, a high water content increases the tanning intensity; differences in the shade depend on the pH; liposomes as carriers act as tanning enhancers.

The intensity of the coloration is also dependent on the thickness of the horny layer. Highly keratinized areas such as the palms of the hands and soles of the feet are colored to a greater degree. The face adopts a lesser shade than the extremities, although the individual differences are very great. In 10 to 15 percent of people, the skin is not colored at all with DHA as a result of a particular skin constitution.

With certain endogenous products, such as tryptophan and alanine, DHA leads to a more yellow dye; particularly in the case of pale-skinned people with poor circulation, the color often looks yellow-tinged. Attempts have therefore been made to mix a plurality of substances (e.g. dihydroxyacetone with erythrulose) in order to achieve an appealing shade of brown. Cosmetically appealing, deep-brown shades were achieved using a combination of dihydroxyacetone and mucondialdehyde. The combination of meso-tartardialdehyde with hydroquinone is also suggested.

As could be shown on histological sections, only the uppermost layers of the horny layer take the dye. Tanning starts after about two to four hours, with repeat application after two to three hours leading to optimum results. The coloration lasts—depending on the exfoliation rate of the upper horny layers—three to seven days, then fades slowly until it has completely disappeared within five to fifteen days.

Self-tanning preparations with dihydroxyacetone or chemically similarly reacting substances are absolutely harmless. Only the dead cell layer of the uppermost horny layers of the skin is colored, damage to deeper layers of skin is ruled out. Since side-effects do not arise—the substances are nontoxic and sensitizations are extremely rare—there is absolutely no objection to the long-term use of self-tanning preparations. However, a decisive disadvantage of DHA-containing self-tanning preparations is that use with self-tanning agents as the sole pigment carriers is only satisfactory in exceptional cases since the resulting shade barely corresponds to the natural healthy skin color.

It was therefore an object of the present invention to arrive, in a simple and cost-effective manner, at self-tanning preparations (in particular at O/W formulations) which are characterized in that the shade achieved with them is as close as possible to the natural skin shade.

It was surprising and unforeseeable by the person skilled in the art that cosmetic or dermatological self-tanning preparations which comprise dihydroxy acetone and are characterized in that they also comprise more than 5% by weight (based on the total weight of the preparation) of glycerin, would overcome the disadvantages of the prior art.

The preparations according to the present invention are extremely satisfactory preparations in every respect which are not limited to a restrictive choice of raw materials. They exhibit very good sensory and cosmetic properties, such as, for example, spreadability on the skin or the ability to absorb into the skin coupled with excellent skincare data. The preparations according to the present invention are further characterized in particular in that the shade of skin achieved with them is more natural than that which can be achieved with comparable products with a lower glycerin content.

The present invention further provides the
use of glycerin in cosmetic or dermatological self-tanning preparations which comprise dihydroxyacetone for achieving naturalness of the skin shade which is formed by the preparation
   and the
use of cosmetic or dermatological self-tanning preparations which comprise dihydroxyacetone and also more than 5% by weight (based on the total weight of the preparation) of glycerin for achieving a natural skin shade.

Within the meaning of the present invention, “natural shade” and “naturalness of the skin shade” are to be understood as meaning that the tan obtained using the self-tanning preparations is comparable with the skin tan or the skin shade which is achieved through gentle suntanning. Whether a skin shade is to be termed natural can be determined, for example, by ascertaining the Natural Tanning Factor (NTF).

To describe a natural tan and to determine the Natural Tanning Factor (NTF), a standard commercial spectrometer is used to record reflection spectra of the skin on the pale inner arm and on the suntanned outer arm by means of light guides.

The spectrum of the outer arm is divided, wavelength-wise, by the spectrum of the inner arm, i.e. standardized. For further evaluation, the standardized spectrum is then used in each case.

If the standardized spectra for the untreated skin are now compared with those which were recorded 24 hours after application of a self-tanning preparation, the tan can be described as more natural, the more alike the shape and progress of the curves.

The NTF corresponds to the ratio of the differences (as a measure of the increase in the spectra within these ranges) of the intensity values at 410 nm (Y₄₁₀) minus that at 500 nm (Y₅₀₀) and the intensity values at 620 nm (Y₆₂₀) and 750 nm (Y₇₅₀):
NTF=(Y₄₁₀−Y₅₀₀)/(Y₆₂₀−Y₇₅₀)

Whether a self-tanning preparation produces a natural tan can thus be ascertained by means of a statistical test in which the individual NTFs of suntanned skin are compared with those of skin tanned with the help of self-tanning preparation of the same group of subjects. If there is no statistically relevant difference between the two sets of data, the tans are described as equal and, accordingly, the artificial tan can be regarded as natural-looking.

It goes without saying that the evaluation of such a test can also be carried out purely visually—for example by the subject himself—or with the help of another measurement method by which color impressions can be ascertained. In each case, however, it should be taken into consideration that—as already explained—in 10 to 15 percent of people, the skin is not colored at all with DHA. In this case, the presented test can of course not be evaluated.

Particularly advantageous preparations within the meaning of the present invention comprise more than 8% by weight (based on the total weight of the preparation) of glycerin.

It is advantageous within the meaning of the present invention to choose the weight ratio of dihydroxyacetone to glycerin to be less than 1, particularly advantageously from 0.05 to 0.9.

It is also preferred if the preparations according to the invention are O/W emulsions, in particular O/W emulsions, which have a droplet size of the internal phase of more than 500 nm, particularly preferably of more than 1000 nm.

O/W emulsions according to the invention preferably comprise one or more emulsifiers which are suitable for producing preparations of this type.

The emulsifier or the emulsifiers are preferably chosen from the group of polyethoxylated fatty acid esters with a chain length of from 10 to 30 carbon atoms and with a degree of ethoxylation of from 5 to 100. For example, polyethoxylated stearic acid esters and polyethoxylated castor oil are particularly advantageous.

The emulsifier or the emulsifiers are also advantageously chosen from the group of mixed esters of saturated, unbranched fatty acids with monomethoxylated glucose and (optionally partially esterified) polyglycerins (e.g. polyglyceryl-3 methylglucose distearate). Advantageous emulsifiers for producing O/W emulsions within the meaning of the present invention are also sodium cetearyl sulfate, and glyceryl stearate, glyceryl isostearate, glyceryl diisostearate, glyceryl oleate, glyceryl palmitate, glyceryl myristate, glyceryl lanolate and/or glyceryl laurate.

It may also be advantageous within the meaning of the present invention to use, in addition to the emulsifier or the emulsifiers, one or more coemulsifiers from the group of fatty alcohols with a chain length of from 10 to 40 carbon atoms for producing an O/W emulsion according to the invention. A particularly preferred coemulsifier is cetearyl alcohol.

Within the meaning of the present invention, self-foaming, foam-like, after-foaming or foamable cosmetic and dermatological preparations are particularly advantageous.

“Self-foaming”, “foam-like”, “after-foaming” and “foamable” preparations are understood as meaning those from which foams can in principle be produced by introducing one or more gases—whether during the preparation process, whether upon use by the consumer or in another way. In such foams, the gas bubbles are (randomly) distributed in one (or more) liquid phase(s), where the (foamed) preparations do not necessarily have the appearance of a foam in macroscopic terms. (Foamed) cosmetic or dermatological preparations according to the invention (referred to below for the sake of simplicity also as foams) may, for example, be macroscopically visible dispersed systems of gases dispersed in liquids. The foam character may, however, for example also only be visible under a (light) microscope. Moreover, foams according to the invention—particularly when the gas bubbles are too small to be seen under a light microscope—are also evident from the considerable volume increase of the system.

Within the meaning of the present invention, such preparations advantageously comprise an emulsifier system which consists of
A. at least one emulsifier A chosen from the group of completely neutralized, partially neutralized or unneutralized branched and/or unbranched, saturated and/or unsaturated fatty acids with a chain length of from 10 to 40 carbon atoms,
B. at least one emulsifier B chosen from the group of polyethoxylated fatty acid esters with a chain length of from 10 to 40 carbon atoms and with a degree of ethoxylation of from 5 to 100 and
C. at least one coemulsifier C chosen from the group of saturated and/or unsaturated, branched and/or unbranched fatty alcohols with a chain length of from 10 to 40 carbon atoms.

The emulsifier or the emulsifiers A are preferably chosen from the group of fatty acids, which are completely or partially neutralized with customary alkalis (such as, for example, sodium hydroxide and/or potassium hydroxide, sodium carbonate and/or potassium carbonate, and mono- and/or triethanolamine). Stearic acid and stearates, isostearic acid and isostearates, palmitic acid and palmitates, and myristic acid and myristates, for example, are particularly advantageous.

The emulsifier or the emulsifiers B are preferably chosen from the following group: PEG-9 stearate, PEG-8 distearate, PEG-20 stearate, PEG-8 stearate, PEG-8 oleate, PEG-25 glyceryl trioleate, PEG-40 sorbitan lanolate, PEG-15 glyceryl ricinoleate, PEG-20 glyceryl stearate, PEG-20 glyceryl isostearate, PEG-20 glyceryl oleate, PEG-20 stearate, PEG-20 methylglucose sesquistearate, PEG-30 glyceryl isostearate, PEG-20 glyceryl laurate, PEG-30 stearate, PEG-30 glyceryl stearate, PEG-40 stearate, PEG-30 glyceryl laurate, PEG-50 stearate, PEG-100 stearate, PEG-150 laurate. Polyethoxylated stearic acid esters, for example, are particularly advantageous.

According to the invention, the coemulsifier or the coemulsifiers C are preferably chosen from the following group: behenyl alcohol (C₂₂H₄₅OH), cetearyl alcohol [a mixture of cetyl alcohol (C₁₆H₃₃OH) and stearyl alcohol (C₁₈H₃₇OH)], lanolin alcohols (wool wax alcohols which are the unsaponifiable alcohol fraction of wool wax which is obtained following saponification of wool wax). Particular preference is given to cetyl and cetylstearyl alcohol.

It is advantageous according to the invention to choose the weight ratios of emulsifier A to emulsifier B to emulsifier C (A:B:C) as a:b:c, where a, b and c, independently of one another, may be rational numbers from 1 to 5, preferably from 1 to 3. A weight ratio of, for example, 1:1:1 is particularly preferred.

It is advantageous within the meaning of the present invention to choose the total amount of the emulsifiers A and B and of the coemulsifier C from the range from 2 to 20% by weight, advantageously from 5 to 15% by weight, in particular from 7 to 13% by weight, in each case based on the total weight of the formulation. According to the invention, it is also particularly preferred if such foam formulations are free from mono- or diglyceryl fatty acid esters. Particular preference is given to foam formulations which comprise no glyceryl stearate, glyceryl isostearate, glyceryl diisostearate, glyceryl oleate, glyceryl palmitate, glyceryl myristate, glyceryl lanolate and/or glyceryl laurate since these compounds adversely affect the foam quality and/or destroy the foam.

The cosmetic or dermatological preparations according to the invention can have the customary composition and serve for the treatment and/or care of the skin and as make-up product in decorative cosmetics.

For use, the preparations according to the invention are applied to the skin in an adequate amount in the manner customary for cosmetics.

The cosmetic and dermatological preparations according to the invention can comprise cosmetic auxiliaries as are customarily used in such preparations, e.g. preservatives, preservative aids, complexing agents, bactericides, perfumes, substances for preventing or increasing foaming, pigments which have a coloring effect, thickeners, moisturizing and/or humectant substances, fillers which improve the feel on the skin, fat, oils, waxes or other customary constituents of a cosmetic or dermatological formulation, such as alcohols, polyols, polymers, foam stabilizers, electrolytes, organic solvents or silicone derivatives.

Advantageous preservatives within the meaning of the present invention are, for example, formaldehyde donors (such as, for example, DMDM hydantoin, which is available, for example, under the trade name Glydant™ from Lonza), iodopropyl butylcarbamates (e.g. those available under the trade names Glycacil-L, Glycacil-S from Lonza, and/or Dekaben LMB from Jan Dekker), parabens (i.e. p-hydroxybenzoic acid alkyl esters, such as methyl- ethyl-, propyl- and/or butylparaben), phenoxyethanol, ethanol, benzoic acid and the like. In addition, the preservative system according to the invention also usually advantageously comprises preservative aids, such as, for example, octoxyglycerin, glycine soya etc.

Within the meaning of the present invention, advantageous complexing agents are, for example, EDTA, [S,S]-ethylenediamine disuccinate (EDDS), which is available, for example, under the trade name Octaquest from Octel, pentasodium ethylenediamine tetramethylenephosphonate, which is available, for example, under the trade name Dequest 2046 from Monsanto and/or diiminosuccinic acid, which is available, inter alia, from Bayer AG under the trade names Iminodisuccinate VP OC 370 (about 30% strength solution) and Baypure CX 100 solid.

Particularly advantageous preparations are also obtained if antioxidants are used as additives or active ingredients. According to the invention, the preparations advantageously comprise one or more antioxidants. Favorable, but nevertheless optional, antioxidants which may be used are all antioxidants customary or suitable for cosmetic and dermatological applications.

Within the meaning of the present invention, water-soluble antioxidants can be used particularly advantageously, such as, for example, vitamins, e.g. ascorbic acid and derivatives thereof.

Preferred antioxidants are also vitamin E and derivatives thereof, and vitamin A and derivatives thereof.

The amount of antioxidants (one or more compounds) in the preparations is preferably 0.001 to 30% by weight, particularly preferably 0.05 to 20% by weight, in particular 0.1 to 10% by weight, based on the total weight of the preparation.

If vitamin E and/or derivatives thereof are the antioxidant or the antioxidants, it is advantageous to choose their respective concentrations from the range from 0.001 to 10% by weight, based on the total weight of the formulation.

If vitamin A or vitamin A derivatives, or carotenes or derivatives thereof are the antioxidant or the antioxidants, it is advantageous to choose their respective concentrations from the range from 0.001 to 10% by weight, based on the total weight of the formulation.

It is particularly advantageous if the cosmetic preparations according to the present invention comprise cosmetic or dermatological active ingredients, preferred active ingredients being antioxidants which can protect the skin against oxidative stress.

Further advantageous active ingredients within the meaning of the present invention are natural active ingredients and/or derivatives thereof, such as, for example, alpha-lipoic acid, phytoene, D-biotin, coenzyme Q10, alpha-glucosylrutin, carnitine, carnosine, natural and/or synthetic isoflavonoids, creatine, taurine and/or β-alanine, and 8-hexadecene-1,16-dicarboxylic acid (dioic acid, CAS number 20701-68-2; provisional INCI name Octadecenedioic acid).

Formulations according to the invention which comprise, for example, known antiwrinkle active ingredients, such as flavone glycosides (in particular α-glycosyirutin), coenzyme Q10, vitamin E and/or derivatives and the like are particularly advantageously suitable for the prophylaxis and treatment of cosmetic or dermatological changes in the skin, as arise, for example, during skin aging (such as, for example, dryness, roughness and formation of dryness wrinkles, itching, reduced regressing (e.g. after washing), visible vascular dilations (teleangiectases, couperosis), flaccidity and formation of lines and wrinkles, local hyperpigmention, hypopigmentation and incorrect pigmentation (e.g. age spots), increased susceptibility to mechanical stress (e.g. cracking) and the like). In addition, they are also advantageously suitable for countering the appearance of dry or rough skin.

The water phase of the preparations according to the present invention can advantageously comprise customary cosmetic auxiliaries, such as, for example, alcohols, in particular those of low carbon number, preferably ethanol and/or isopropanol, polymers, foam stabilizers, electrolytes, and in particular one or more thickeners, which may advantageously be chosen from the group consisting of silicon dioxide, aluminum silicates, polysaccharides or derivatives thereof, e.g. hyaluronic acid, xanthan gum, hydroxypropylmethylcellulose, particularly advantageously from the group of polyacrylates, preferably a polyacrylate from the group of so-called carbopols [from Noveon], for example carbopol grades 980, 981, 1382, 2984, 5984, ETD 2020, ETD 2050, Pemulen TR-1 or -2, Ultrez 10, in each case individually or in combination.

In addition, the preparations according to the present invention can advantageously also comprise repellents for protection against flies, ticks and spiders and the like. For example, N,N-diethyl-3-methylbenzamide (trade name: metadelphene, “DEET”), dimethyl phthalate (trade name: Palatinol M, DMP), and in particular ethyl 3-(N-n-butyl-N-acetylamino)propionate (available under the trade name Insekt Repellent® 3535 from Merck) are advantageous. The repellents can either be used individually or in combination.

Moisturizers is the term used to refer to substances or mixtures of substances which impart to cosmetic or dermatological preparations the property, following application or distribution on the surface of the skin, of reducing moisture release from the horny layer (also called trans-epidermal water loss (TEWL)) and/or of positively influencing hydration of the horny layer.

Although glycerin is itself a moisturizer, it may be advantageous within the meaning of the present invention to use further substances from this group, for example lactic acid and/or lactates, in particular sodium lactate, butylene glycol, propylene glycol, biosaccharide gum-1, glycine soya, ethylhexyloxyglycerin, pyrrolidonecarboxylic acid and urea. In addition, it is particularly advantageous to use polymeric moisturizers from the group of water-soluble and/or water-swellable and/or water-gellable polysaccharides. Hyaluronic acid, chitosan and a fucose-rich polysaccharide, which is filed in the Chemical Abstracts under the Registry number 178463-23-5 and which is available, for example, under the name Fucogel® 1000 from SOLABIA S. A., for example, are particularly advantageous. Moisturizers can advantageously also be used as antiwrinkle active ingredients for the prophylaxis and treatment of cosmetic or dermatological changes in the skin, as arise, for example, during skin aging.

The cosmetic or dermatological preparations according to the invention can also advantageously, but not necessarily, comprise fillers which, for example, further improve the sensory and cosmetic properties of the formulations and, for example, bring about or enhance a velvety or silky feel on the skin. Advantageous fillers within the meaning of the present invention are starch and starch derivatives (such as, for example, tapioca starch, distarch phosphate, aluminum or sodium starch octenylsuccinate and the like), pigments which have neither a primary UV filter effect nor a coloring effect (such as, for example, boron nitride etc.) and/or Aerosils® (CAS No. 7631-86-9).

The oil phase of the formulations according to the invention is advantageously chosen from the group of polar oils, for example from the group of lecithins and fatty acid triglycerides, namely the triglycerol esters of saturated and/or unsaturated, branched and/or unbranched alkanecarboxylic acids of chain length from 8 to 24, in particular 12 to 18 carbon atoms. The fatty acid triglycerides can, for example, be chosen advantageously from the group of synthetic, semisynthetic and natural oils, such as, for example, cocoglyceride, olive oil, sunflower oil, soya oil, peanut oil, rapeseed oil, almond oil, palm oil, coconut oil, castor oil, wheatgerm oil, grapeseed oil, thistle oil, evening primrose oil, macadamian nut oil and the like.

Also advantageous according to the invention are, for example, natural waxes of animal and vegetable origin, such as, for example, beeswax and other insect waxes, and berry wax, shea butter and/or lanolin (wool wax).

Further advantageous polar oil components can, within the meaning of the present invention, also be chosen from the group of esters of saturated and/or unsaturated, branched and/or unbranched alkane carboxylic acids of chain length from 3 to 30 carbon atoms and saturated and/or unsaturated, branched and/or unbranched alcohols of chain length from 3 to 30 carbon atoms, and from the group of esters of aromatic carboxylic acids and saturated and/or unsaturated, branched and/or unbranched alcohols of chain length from 3 to 30 carbon atoms. Such ester oils can then advantageously be chosen from the group consisting of octyl palmitate, octyl cocoate, octyl isostearate, octyldodecyl myristate, octyldodecanol, cetearyl isononanoate, isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, stearyl heptanoate, oleyl oleate, oleyl erucate, erucyl oleate, erucyl erucate, tridecyl stearate, tridecyl trimellitate, and synthetic, semisynthetic and natural mixtures of such esters, such as, for example, jojoba oil.

In addition, the oil phase can advantageously be chosen from the group of dialkyl ethers and dialkyl carbonates, advantageous examples being dicaprylyl ether (Cetiol OE) and/or dicaprylyl carbonate, for example that available under the trade name Cetiol CC from Cognis.

It is also preferred to choose the oil component or the oil components from the group consisting of isoeicosane, neopentyl glycol diheptanoate, propylene glycol dicaprylate/dicaprate, caprylic/capric/diglyceryl succinate, butylene glycol dicaprylate/dicaprate, C₁₂₋₁₃-alkyl lactate, di-C₁₂₋₁₃-alkyl tartrate, triisostearin, dipentaerythrityl hexacaprylate/hexacaprate, propylene glycol monoisostearate, tricaprylin, dimethyl isosorbide. It is particularly advantageous if the oil phase of the formulations according to the invention has a content of C₁₂₋₁₅-alkyl benzoate or consists entirely of this.

Advantageous oil components are also, for example, butyloctyl salicylate (for example that available under the trade name Hallbrite BHB from CP Hall), hexadecyl benzoate and butyloctyl benzoate and mixtures thereof (Hallstar AB) and/or diethylhexyl naphthalate (Hallbrite TQ or Corapan TQ from Symrise).

Any mixtures of such oil and wax components can also be used advantageously within the meaning of the present invention.

Furthermore, the oil phase can likewise advantageously also comprise nonpolar oils, for example those which are chosen from the group of branched and unbranched hydrocarbons and hydrocarbon waxes, in particular mineral oil, Vaseline (petrolatum), paraffin oil, squalane and squalene, polyolefins, hydrogenated polyisobutenes and isohexadecane. Among the polyolefins, polydecenes are the preferred substances.

Advantageously, the oil phase can also have a content of cyclic or linear silicone oils or consist entirely of such oils, although it is preferred to use an additional content of other oil phase components apart from the silicone oil or the silicone oils.

Silicone oils are high molecular weight synthetic polymeric compounds in which silicon atoms are joined via oxygen atoms in a chain-like or grid-like manner and the remaining valences of the silicon are saturated by hydrocarbon radicals (in most cases methyl groups, more rarely ethyl, propyl, phenyl groups etc.). Systematically, the silicone oils are referred to as polyorganosiloxanes. The methyl-substituted polyorganosiloxanes, which represent the most significant compounds of this group in terms of amount and are characterized by the following structural formula 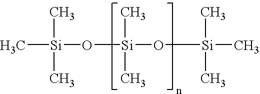
are also referred to as polydimethylsiloxane or dimethicone (INCI). Dimethicones come in various chain lengths and with various molecular weights.

Particularly advantageous polyorganosiloxanes within the meaning of the present invention are, for example, dimethylpolysiloxanes [poly(dimethylsiloxane)], which are available, for example, under the trade names Abil 10 to 10 000 from Th. Goldschmidt. Also advantageous are phenylmethylpolysiloxanes (INCI: Phenyl Dimethicone, Phenyl Trimethicone), cyclic silicones (octamethylcyclotetrasiloxane and decamethylcyclopentasiloxane), which are also referred to as Cyclomethicones in accordance with INCI, amino-modified silicones (INCI: Amodimethicones) and silicone waxes, e.g. polysiloxane-polyalkylene copolymers (INCI: Stearyl Dimethicone and Cetyl Dimethicone) and dialkoxydimethylpolysiloxanes (Stearoxy Dimethicone and Behenoxy Stearyl Dimethicone), which are available as various Abil wax grades from Th. Goldschmidt. Other silicone oils can, however, also be used advantageously within the meaning of the present invention, for example cetyldimethicone, hexamethylcyclotrisiloxane, polydimethylsiloxane, poly(methylphenylsiloxane).

It is preferred according to the invention to choose the content of UV filter substances (one or more compounds) to be less than 5% by weight, in particular less than 2% by weight, in each case based on the total weight of the preparation. Within the meaning of the present invention, preparations which are free from UV filter substances are very particularly advantageous.

If a content of UV filter substances is desired, these are advantageously chosen from one or more of the following groups: UV-A, UV-B and/or broadband filter substances, and organic and/or inorganic pigments as UV filter substances.

Particularly advantageous UV filter substances within the meaning of the present invention are:
dibenzoylmethane derivatives, in particular 4-(tert-butyl)-4′-methoxydibenzoylmethane (CAS No. 70356-09-1), which is sold by Givaudan under the name Parsol® 1789 and by Merck under the trade name Eusolex® 9020.
phenylene-1,4-bis(2-benzimidazyl)-3,3′-5,5′-tetrasulfonic acid and its salts, particularly the corresponding sodium, potassium or triethanolammonium salts, in particular the phenylene-1,4-bis(2-benzimidazyl)-3,3′-5,5′-tetrasulfonic acid bis-sodium salt with the INCI name Disodium Phenyl Dibenzimidazol Tetrasulfonate (CAS No.: 180898-37-7), which is available, for example, under the trade name Neo Heliopan AP from Symrise;
salts of 2-phenylbenzimidazole-5-sulfonic acid, such as its sodium, potassium or its triethanolammonium salt, and the sulfonic acid itself with the INCI name Phenylbenzimidazole Sulfonic Acid (CAS No. 27503-81-7), which is available, for example, under the trade name Eusolex 232 from Merck or under Neo Heliopan Hydro from Symrise;
1,4-di(2-oxo-10-sulfo-3-bornylidenemethyl)benzene (also: 3,3′-(1,4-phenylenedimethylene)bis(7,7-dimethyl-2-oxobicyclo[2.2.1]hept-1-ylmethanesulfonic acid) and salts thereof (particularly the corresponding 10-sulfato compounds, in particular the corresponding sodium, potassium or triethanolammonium salt), which is also referred to as benzene-1,4-di(2-oxo-3-bornylidenemethyl-10-sulfonic acid). Benzene-1,4-di(2-oxo-3-bornylidenemethyl-10-sulfonic acid) has the INCI name Terephthalidene Dicamphor Sulfonic Acid (CAS No.: 90457-82-2) and is available, for example, under the trade name Mexoryl SX from Chimex;
sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and salts thereof.
benzoxazole derivatives, such as, for example, 2,4-bis[5-1(dimethylpropyl)benz-oxazol-2-yl(4-phenyl)imino]-6-(2-ethylhexyl)imino-1,3,5-triazine with the CAS No. 288254-16-0, which is available from 3V Sigma under the trade name Uvasorb® K2A.
hydroxybenzophenones, e.g. hexyl 2-(4′-diethylamino-2′-hydroxybenzoyl)benzoate (also: aminobenzophenone), which is available under the trade name Uvinul A Plus from BASF.
triazine derivatives, such as, for example, 2,4-bis{[4-(2-ethylhexyloxy)-2-hydroxyl]phenyl}-6-(4-methoxyphenyl)-1,3,5-triazine (INCI: Bis-Ethylhexyloxyphenol Methoxyphenyl Triazine), which is available under the trade name Tinosorb® S from CIBA-Chemikalien GmbH; dioctylbutylamidotriazone (INCI: Diethylhexyl Butamido Triazone), which is available under the trade name UVASORB HEB from Sigma 3V; tris(2-ethylhexyl) 4,4′,4″-(1,3,5-triazine-2,4,6-triyltriimino)trisbenzoate, also: 2,4,6-tris[anilino(p-carbo-2′-ethyl-1′-hexyloxy)]-1,3,5-triazine (INCI: Ethylhexyl Triazone), which is sold by BASF Aktiengesellschaft under the trade name UVINUL® T 150; 2-[4,6-bis(2,4-dimethylphenyl)-1,3,5-triazine-2-yl]-5-(octyloxy)phenol (CAS No.: 2725-22-6).
benzotriazoles, such as, for example, 2,2′-methylenebis(6-(2H-benzotriazol-2-yl)-4-(1,1,3,3-tetramethylbutyl)phenol) (INCI: Methylene Bis-Benztriazolyl Tetramethylbutylphenol), which is available, for example, under the trade name Tinosorb® M from CIBA-Chemikalien GmbH.
3-benzylidenecamphor derivatives, preferably 3-(4-methylbenzylidene)camphor, 3-benzylidenecamphor;
4-aminobenzoic acid derivatives, preferably 2-ethylhexyl 4-(dimethylamino)benzoate, amyl 4-(dimethylamino)benzoate;
esters of benzalmalonic acid, preferably di(2-ethylhexyl) 4-methoxybenzalmalonate;
esters of cinnamic acid, preferably 2-ethylhexyl 4-methoxycinnamate, isopentyl 4-methoxycinnamate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone and
UV filters bonded to polymers
ethylhexyl 2-cyano-3,3-diphenylacrylate (octocrylene), which is available from BASF under the name Uvinul® N 539 T.

The examples below serve to illustrate the present invention without limiting it. The numerical values in the examples are percentages by weight, based on the total weight of the respective preparations.

### EXAMPLES

| | 1 | 2 | 3 | 4 | 5 | 6 |
|---|---|---|---|---|---|---|
| | Lotion | Cream | Lotion | Foam | Cream | Lotion |
| Dihydroxyacetone | 3 | 5 | 4 | 2 | 3 | 5 |
| Glycerin | 6 | 12 | 8 | 9 | 10 | 12 |
| Polyglyceryl-3 methylglucose | 4.5 | | | | | |
| distearate | | | | | | |
| Sodium cetearyl sulfate | | | 1 | | | |
| PEG-40 stearate | | 3 | | 2.5 | | |
| PEG-40 castor oil | | | 1 | | | |
| Cetearyl alcohol | | | 2 | | | |
| Cetyl alcohol | 1 | 3 | | 2.5 | | |
| Stearyl alcohol | | 1 | | | 2 | |
| Sorbitan stearate | | | | 1 | | |
| Glyceryl stearate | | 2 | | | | |
| Glyceryl stearate SE | | | 2 | | | |
| Glyceryl stearate citrate | | | | | 3 | 1 |
| Myristyl myristate | 2 | 3 | | | 1 | |
| Myristyl lactate | | 2 | | | 2 | |
| Stearic acid | | | | 2.5 | | |
| Alcohol | | | 4 | | 4 | 3 |
| Silicone oil | 7 | | 4 | | 2 | 4 |
| C12-15 Alkyl benzoate | 2 | | | | 2 | |
| Caprylic/capric triglyceride | 2 | 3 | | | | |
| Cetyl ricinoleate | | | | 1.5 | | 1 |
| Octyldodecanol | 3 | 4 | 2 | | 2 | |
| Mineral oil | | 2 | 5 | | | 1 |
| Macadamia nut oil | 2 | 1 | 3 | | 3 | 1 |
| Dicaprylyl carbonate | 2 | | | 1 | | |
| Butyl | | 3 | | | 1 | |
| methoxydibenzoylmethane | | | | | | |
| bis-Ethylhexyloxyphenol triazine | | | | | 2 | |
| Sorbitol | | 2 | | | | 2 |
| Butylene glycol | | | 2 | | | |
| EDTA | 1 | 1 | 1 | 1.5 | 2 | 1 |
| Xanthan gum | 0.5 | 0.2 | 0.3 | | | 1 |
| C10-30 alkyl acrylate | | | | | | 0.3 |
| crosspolymer | | | | | | |
| Sodium hydroxide solution | | | | 0.1 | | 0.1 |
| Sodium citrate | | | 0.4 | | 0.3 | |
| Citric acid | 0.15 | 0.15 | 0.3 | | 0.2 | 0.3 |
| Vitamin E acetate | 1 | 0.5 | 0.5 | | 1 | 1 |
| Phenoxyethanol | 0.4 | | 0.4 | 0.5 | | 0.4 |
| Methylparaben | 0.3 | | | 0.3 | 0.2 | |
| Ethylparaben | 0.1 | | | 0.1 | | |
| DMDM hydantoin | | | 0.1 | | | |
| Iodopropynyl butylcarbamate | | 0.15 | | 0.1 | 0.2 | 0.2 |
| Perfume | 0.4 | | 0.3 | 0.2 | 0.3 | |
| Water | ad | ad | ad | ad | ad | ad 100 |
| | 100 | 100 | 100 | 100 | 100 | |

The foam can be bottled either as pump foamer in a suitable dispensing system, or else in aerosol containers mixed with propane/isobutane or propane/butane (in each case 3:7) in a bottling ratio of concentrate:propellant gas 97:3 to 80:10.

The lotions can be used as such or else for use on wipes.

| | 7 | 8 | 9 | 10 |
|---|---|---|---|---|
| | Cream | Lotion | Lotion | Lotion |
| Dihydroxyacetone | 5 | 3 | 4 | 6 |
| Glycerin | 10 | 8 | 6 | 12 |
| Cetearyl alcohol | | 2 | | |
| Behenyl alcohol | 1 | | | 1 |
| Arachidyl alcohol | 3 | | | |
| PPG-20 methyl glucose ether | | | 1 | |
| distearate | | | | |
| Polysorbate-20 | | | 3 | |
| Methylglucose sesquistearate | | 2 | | |
| PEG-100 stearate | 3 | | | |
| Dimethicone copolyol | | | | 1.5 |
| Glyceryl stearate | 2 | | | |
| Alcohol | | | 3 | |
| Silicone oil | 2 | 4 | | |
| C12-15 Alkyl benzoate | 5 | | | |
| Caprylic/capric triglyceride | | | 3 | |
| Octyldodecanol | | 3 | | 2 |
| Mineral oil | | | | 2 |
| Macadamia nut oil | 1 | | 2 | |
| Dicaprylyl carbonate | | 2 | | |
| Butyl methoxydibenzoylmethane | | | 3 | |
| Bis-ethylhexyloxyphenol triazine | | 2 | | |
| Sorbitol | | 4 | | |
| Butylene glycol | 3 | | | |
| EDTA | 1 | 1 | 1.5 | 2 |
| Xanthan gum | 0.2 | 0.3 | | |
| Polyacrylamide | 1 | | 1 | |
| Sodium hydroxide solution | 0.1 | | 0.1 | |
| Sodium citrate | | 0.4 | | 0.3 |
| Citric acid | 0.15 | 0.3 | | 0.2 |
| Lactic acid | | | 0.3 | |
| Vitamin E acetate | 0.5 | 0.5 | | 1 |
| Phenoxyethanol | 0.1 | 0.4 | 0.5 | |
| Methylparaben | 0.3 | | 0.3 | 0.2 |
| Ethylparaben | | | 0.1 | |
| DMDM hydantoin | | 0.1 | | |
| Iodopropynyl butylcarbamate | 0.15 | | 0.1 | 0.2 |
| Perfume | | 0.3 | 0.2 | 0.3 |
| Water | ad | ad | ad | ad |
| | 100 | 100 | 100 | 100 |

1.-10. (canceled)

11. A cosmetic or dermatological self-tanning composition which comprises dihydroxyacetone and more than 5% by weight, based on a total weight of the composition, of glycerin.

12. The composition of claim 11, wherein the composition comprises more than 8% by weight of glycerin.

13. The composition of claim 11, wherein a weight ratio of dihydroxyacetone and glycerin is smaller than 1:1.

14. The composition of claim 13, wherein the weight ratio is from 0.05:1 to 0.9:1.

15. The composition of claim 11, wherein the composition further comprises one or more O/W emulsifiers.

16. The composition of clam 15, wherein the one or more O/W emulsifiers comprise at least one emulsifier selected from polyethoxylated esters of fatty acids having a chain length of from 10 to 30 carbon atoms and a degree of ethoxylation of from 5 to 100 and esters of saturated, unbranched fatty acids with monomethoxylated glucose or polyglycerols.

17. The composition of claim 16, wherein the one or more OAN emulsifiers comprises at least one of a polyethoxylated ester of stearic acid and a polyethoxylated castor oil.

18. The composition of clam 15, wherein the one or more O/W emulsifiers comprise at least one emulsifier selected from sodium cetearyl sulfate, glyceryl stearate, glyceryl isostearate, glyceryl diisostearate, glyceryl oleate, glyceryl palmitate, glyceryl myristate, glyceryl lanolate and glyceryl laurate.

19. The composition of claim 15, wherein the composition further comprises at least one coemulsifier selected from fatty alcohols having a chain length of from 10 to 40 carbon atoms.

20. The composition of claim 19, wherein the at least one coemulsifier comprises cetearyl alcohol.

21. The composition of claim 11, wherein the composition comprises less than 5% by weight of one or more UV filter substances, based on the total weight of the composition.

22. The composition of claim 11, wherein the composition comprises more than 8% by weight of glycerin, a weight ratio of dihydroxyacetone and glycerin is smaller than 1:1, and the composition further comprises one or more O/W emulsifiers selected from polyethoxylated esters of fatty acids having a chain length of from 10 to 30 carbon atoms and a degree of ethoxylation of from 5 to 100, esters of saturated, unbranched fatty acids with monomethoxylated glucose or polyglycerols, and sodium cetearyl sulfate, glyceryl stearate, glyceryl isostearate, glyceryl diisostearate, glyceryl oleate, glyceryl palmitate, glyceryl myristate, glyceryl lanolate and glyceryl laurate.

23. The composition of claim 22, wherein the composition further comprises at least one coemulsifier selected from fatty alcohols having a chain length of from 10 to 40 carbon atoms.

24. The composition of claim 23, wherein the weight ratio of dihydroxyacetone and glycerin is from 0.05:1 to 0.9:1.

25. A cosmetic or dermatological self-tanning composition which comprises dihydroxyacetone and glycerin in a weight ratio of from 0.05:1 to 0.9:1.

26. The composition of claim 25, wherein the composition comprises more than 8% by weight of glycerin.

27. The composition of claim 25, wherein the composition further comprises one or more O/W emulsifiers.

28. The composition of clam 27, wherein the one or more O/W emulsifiers comprise at least one emulsifier selected from polyethoxylated esters of fatty acids having a chain length of from 10 to 30 carbon atoms and a degree of ethoxylation of from 5 to 100, esters of saturated, unbranched fatty acids with monomethoxylated glucose or polyglycerols, and sodium cetearyl sulfate, glyceryl stearate, glyceryl isostearate, glyceryl diisostearate, glyceryl oleate, glyceryl palmitate, glyceryl myristate, glyceryl lanolate and glyceryl laurate.

29. An oil-in-water emulsion which comprises the composition of claim 11.

30. The emulsion of claim 29, wherein a droplet size of an inner phase of the emulsion is larger than 500 nm.

31. The emulsion of claim 30, wherein the droplet size is larger than 1,000 nm.

32. A method of providing skin with a natural tanned color, wherein the method comprises applying to skin the composition of claim 11 in an amount which is sufficient to provide a tanned color.

33. A method of producing a dihydroxyacetone containing cosmetic or dermatological self-tanning composition which provides a natural tanned color, wherein the method comprises incorporating into the composition an amount of glycerin which is sufficient for the composition to provide a natural skin color when it is applied to skin.
